(19) United States
(12) Patent Application Publication
(10) Pub. No.: US 2015/0174430 A1
(43) Pub. Date: Jun. 25, 2015

(54) RADIATION DOSE CONTROL DEVICE FOR CONTROLLING AN ELECTRON BEAM PULSE DELIVERED DURING IORT

(71) Applicant: S.I.T.- Sordina IORT Technologies S.P.A., Vicenza (VI) (IT)

(72) Inventors: Giuseppe Felici, Vicenza (VI) (IT); Vincenzo Iacoboni, Vicenza (VI) (IT); Alessia Ciccotelli, Vicenza (VI) (IT); Fabio De Angelis, Vicenza (VI) (IT)

(21) Appl. No.: 14/402,887

(22) PCT Filed: May 22, 2013
(86) PCT No.: PCT/IT2013/000143

§ 371 Date: Nov. 21, 2014

(30) Foreign Application Priority Data
May 22, 2012 (IT) VI2012A000119

(51) Int. Cl. A61N 5/10

(52) U.S. Cl. CPC A61N 5/1071; A61N 5/1077; A61N 2005/1089

(57) ABSTRACT

A radiation dose control device for controlling an electron beam pulse delivered during a therapy session of IORT (Intra-Operative Radiation Therapy), comprising a PWM system configured to provide an electron injection at a DC voltage at each pulse of an input electron beam (FE) sent to the input of an electronic gun (G) of a linear accelerator or linac (AL), so that the output electron beam (FU) exiting said linac (AL) is highly stable, and so that a variation of the radiation dose of said output electron beam (FU) results only from the variation of the delivery time of said input electron beam (FE); said dose variation of the output electron beam (FU) is thus directly proportional to said delivery time of the input electron beam (FE).

The present invention relates to a radiation dose control device for controlling an electron beam pulse delivered during a therapy session of IORT (Intra Operative Radiation Therapy) and irradiated above organs and tissues which are placed downstream said-control device.

The invention also relates to an IORT machine provided with said radiation dose control device. It is well known that intra-operative radiotherapy (IORT) is an innovative technique that is gradually spreading worldwide in the treatment of various type of tumors thanks to the development of mobile machines.

In particular, the intra-operative radiotherapy consists of an irradiation performed during the surgical removal of the tumor mass; said irradiation is generally performed by means of a uniform and widespread electron beam constituting the ionizing particles with a kinetic energy between 4 and 12 MeV. Practically, as a result of the surgical removal of the tumor mass, the surrounding or adjacent tissues near the tumor mass are irradiated with the electron beam in order to damage the tumor cells and to prevent their re-population; when the disease does not present the existence of metastasis, the intra-operative radiation therapy can be applied as part of the total radiation therapy, or as a single irradiation, as for a large category of patients the intra-operative irradiation totally and successfully replaces the known radiation therapy, thus drastically shortening the duration of the therapeutic phase.

The fixed or mobile IORT machines allow irradiation of the target with a substantially cylindrical or elliptical symmetry; said irradiation is provided by applying a metal or plastic tube to the tissues, in order to convey and spread on the target an electron beam generated by an electron accelerator.

In particular, a proximal element of the tube is fixed to the radiating head, while a distal element contacts the area to be irradiated and is fixed to the proximal element.

The electromagnetic field which is obtained with said structure has an obvious cylindrical symmetry, which is adequate in the treatment of some cancers, especially the cancers of the breast; moreover, the healthy tissues, which are placed below the irradiated area, are also protected by a radioprotection disc having a diameter corresponding to the tube's diameter.

Moreover, the repetition frequency of the electron beam is usually varied between 10 and 40 Hz, in order to ensure a dose rate greater than or equal to 10 Gy/min with a tube having a diameter of 100 mm, and it is possible to have higher dose rates, up to 30 Gy/min;

however, it is not possible to establish the ideal dose rate when a single treatment is provided, as well as it is not possible to modulate the dose rate according to suitable radiobiological guidelines.

The main object of the present invention is, therefore, to obviate the drawbacks of the above mentioned prior art and, in particular, to provide a radiation dose control device for controlling an electron beam pulse delivered during IORT, which allows to modulate the dose rate according to the suitable radiobiological guidelines, when a single treatment is performed.

Another object of the invention is to provide a radiation dose control device for controlling an electron beam pulse delivered during IORT, which is configured to provide a modulation of the dose rate and of the radiation dose for each electron beam pulse, in particular by means of a diode-type electronic gun.

Another object of the invention is to provide a radiation dose control device for controlling an electron beam pulse delivered during IORT, which is capable to quickly obtain an ideal value of the electronic radiation dose rate.

A further object of the invention is to provide a radiation dose control device for controlling an electron beam pulse delivered during IORT, which, after having achieved said ideal value, is also configured to set the dose rate of the electronic radiation.

The above mentioned objects are achieved by means of a radiation dose control device for controlling an electron beam pulse delivered during IORT, according to the appended claim 1; other detailed technical features are contained in the dependent claims.

A further object of the present invention is to provided a related measurement method according to the appended claim 8 and an IORT machine which includes the radiation dose control device according to the appended claim 9.

Advantageously, the radiation dose control device according to the invention is configured to provide a dose and a dose rate modulation of the electronic radiation for each electron beam pulse by using a diode-type electronic gun and by using a simple structure, with a simple electronic control circuit, and without using additional high voltage cables.

It is thus possible to modulate the dose rate according to the suitable radiobiological guidelines and it is also possible to obtain an ideal value of said dose rate, which is consequently fixed within the electron beam.

Moreover, it is possible to have any prefixed ratio between the dose rate and the Monitor Unit (UM) of the machine and it is also possible to distinguish the values of the desired dose rate for each value of energy, thus also optimizing the timing of the therapy session.

Finally, it is possible to obtain a ratio between dose and pulse which is constant and which can be repeated over time.

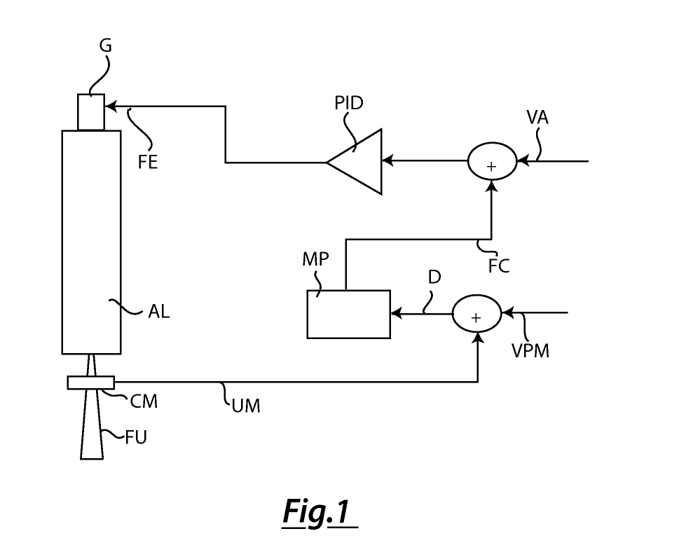

The present invention will now be described according to a preferred embodiment and with particular reference to the enclosed FIG. 1, which schematically shows a block diagram of the radiation dose control device for controlling an electron beam pulse delivered during IORT, which is the object of the present invention.

Referring to the above mentioned FIG. 1, the radiation dose control device for controlling an electron beam pulse delivered during an IORT single session, according to the present invention, comprises a PWM system, which is configured to provide an electron beam pulse having an electron dose rate which is constant over time.

The electronic gun G sends the input electron beam FE to the linear accelerator or linac AL (LINAC), so as to have a DC-type injection of electrons.

Moreover, the input electron beam FE is delayed so as to be injected into the electronic gun G when a maximum RF-energy transfer is provided to the linac AL, thus having electrons inside the linac AL with energy constant over time.

The output electron beam FU exiting the linac AL is therefore highly stable and the dose variation is obtained only because the delivery time of the input electron beam FE (pulse width) is varied; said dose variation is directly proportional to said delivery time.

Since the dose is proportional to the time duration of the electron beam FE, as described above, it is thus possible to easily reduce or increase said dose value and to make the actual value of the machine Monitor Units UM (taken from the machine Monitor Chambers CM placed on the output electron beam FU) equal to a prefixed value VPM in standard units (for example, 1 cGy=1 UM), while a microprocessor control unit MP also allows to distinguish the desired electron beam dose values for each value of energy, thus also optimizing the timing of the therapy session.

Once the current value of Monitor Units UM is settled, as described, a PID feedback control system, which is controlled by the microprocessor control unit MP, is used; a correction factor FC is also added, at the output of said PID system, to the current value VA of the pulse width of the input electron beam FE.

Consequently, a very accurate and time-repeatable ratio between the electron beam dose and the electron beam pulse is obtained.

The radiation dose control device for controlling an electron beam pulse delivered during IORT, according to the present invention, substantially operates as follows.

For each electron beam pulse, the present value of Monitor Units UM is compared with the prefixed value VPM and the difference D between the two signals is used, by means of the microprocessor unit control MP, for amending the duration of the electron beam pulse FE at the input of the electron guy G and at the cathode of the linac AL and for also performing a time pulse width modulation, by means of the PID feedback system, in order to put the current value of Monitor Units UM equal to the prefixed value VPM; the above mentioned method is performed for every available value of energy.

Moreover, as already mentioned, the electron beam pulse which is generated in correspondence of the electronic gun G is delayed, so that the RF transient is over, in order to ensure that the time interval within which the electrons are generated is equal to the time interval within which the RF generator (typically a Magnetron) is performing the maximum transfer of the power to the linac AL; thus, the kinetic energy of the output electron beam FU exiting the linac AL has a constant value.

Finally, the device of the present invention is configured to obtain a suitable modulation of the electron dose rate and of the electron dose for each electron beam pulse exiting from a diode-type electronic gun G, unlike the triode-type electronic guns which were used in the prior art; a further advantage is constituted by the use of a diode-type electronic gun, since a triode-type electronic gun requires a more complex structure, a considerably more complex control circuit and, not least, an additional high voltage cable.

From the above description, the features of the radiation dose control device for controlling an electron beam pulse delivered during IORT, which is the object of the present invention, are clear as well as clear the related advantages.

In particular, said advantages consist of the following characteristics:
modulating the dose rate according to the suitable radiobiological guidelines;
fixing a dose rate of the electron beam;
DC-type electrons injection for each electron beam pulse;
establishing any prefixed values of the dose/Monitor Units ratio;
dose/pulse ratio which is constant over time.

It is finally clear that other variations may be made to the radiation dose control device of the invention, without departing from the principles of novelty inherent in the inventive idea, as it is clear that, during the practical implementation of the invention, materials, shapes and dimensions of the technical features which are shown may be any according to requirements and that they can be replaced with other technically equivalent features, without thereby departing from the scope of protection as defined by the appended claims.

1-9. (canceled)

10. An intra-operative radiotherapy or IORT machine comprising:
a linear accelerator or LINAC (AL);
a diode-type electron gun (G);
a PWM system configured to provide an injection of a first electron beam (FE), with the same DC voltage and energy, from said electron gun (G) into said LINAC (AL), so that a second electron beam (FU) at the output of said LINAC (AL) is highly stable,
characterized in that said IORT machine also comprises a dose control device for controlling the dose, for each pulse, of said second electron beam (FU) which is delivered during a treatment of intra-operative radiotherapy, said dose control device being used for obtaining a dose variation of said second electron beam (FU) only on the basis of a variation of the delivery time of said first electron beam (FE), wherein said dose variation of said second electron beam (FU) is directly proportional to said delivery time of said first electron beam (FE) and wherein said dose control device is configured to delay said first electron beam (FE), which is injected into said LINAC (AL) in a phase during which a maximum amount of energy is transferred to said LINAC (AL).

11. An IORT machine as claimed in claim 10, characterized in that said dose control device includes Monitor Units (MU) and Monitor Chambers (CM), wherein said Monitor Units (MU) are placed at the output of said Monitor Chambers (CM) and said Monitor Chambers (CM) are placed along said second electron beam (FU) at the output of said LINAC (AL) and said dose variation of said second electron beam (FU) is obtained by measuring said Monitoring Units (UM), whose current value is set equal to a prefixed value (VPM) in standard units.

12. An IORT machine as claimed in claim 10, characterized in that said dose control device further comprises a microprocessor control unit (MP), which is used to vary the values of said dose for each value of energy of said LINAC (AL).

13. An IORT machine as claimed in claim 12, characterized in that said dose control device further comprises a PID feedback control system, controlled by said microprocessor control unit (MP), which sends a constant signal to said LINAC (AL), said constant signal being related to the ratio between said variation dose of said second electron beam (FU) and the radiation pulse of said first electron beam (FE) injected into said LINAC (AL), said microprocessor control unit (MP) being configured to provide a correction factor (FC) which is added to a current value (VA) of the pulse width of said first electron beam (FE).

14. A dose control method performed by a dose control device as claimed in claim 12, characterized in that it comprises at least the following phases:
comparing, for each pulse of said first electron beam (FE), a signal corresponding to said current value of Monitor Units (MU) with a signal corresponding to said prefixed value (VPM) in standard units;
using a difference signal (D) between said signal corresponding to said current value of Monitor Units (MU) and said signal corresponding to said prefixed value (VPM) in standard units, in order to rectify, by means of said microprocessor control unit (MP), the pulse duration of said first electron beam (FE) which is injected into said LINAC (AL);
obtaining a pulse-width modulation (PWM), through a PID feedback system, of the signal at the input of said electron gun (G) and of said LINAC (AL), for each value of energy, so as to make said current value of Monitor Units (MU) equal to said prefixed value (VPM) in standard units.

15. An IORT machine as claimed in claim 11, characterized in that said dose control device further comprises a microprocessor control unit (MP), which is used to vary the values of said dose for each value of energy of said LINAC (AL).

16. A dose control method performed by a dose control device as claimed in claim 13, characterized in that it comprises at least the following phases:
comparing, for each pulse of said first electron beam (FE), a signal corresponding to said current value of Monitor Units (MU) with a signal corresponding to said prefixed value (VPM) in standard units;
using a difference signal (D) between said signal corresponding to said current value of Monitor Units (MU) and said signal corresponding to said prefixed value (VPM) in standard units, in order to rectify, by means of said microprocessor control unit (MP), the pulse duration of said first electron beam (FE) which is injected into said LINAC (AL);
obtaining a pulse-width modulation (PWM), through a PID feedback system, of the signal at the input of said electron gun (G) and of said LINAC (AL), for each value of energy, so as to make said current value of Monitor Units (MU) equal to said prefixed value (VPM) in standard units.
